(19) United States
(12) Patent Application Publication
(10) Pub. No.: US 2024/0318128 A1
(43) Pub. Date: Sep. 26, 2024

(54) BIOFUNGICIDE PRODUCTION BY CONIOCHAETA AND APPLICATION IN CONTROL OF EARLY BLIGHT OF POTATO

(71) Applicant: The United States of America, as represented by the Secretary of Agriculture, Washington, DC (US)

(72) Inventors: RICHARD W. JONES, SILVER SPRING, MD (US); CATALINA SALGADO-SALAZAR, BELTSVILLE, MD (US); DIMITRE S. MOLLOV, PHILOMATH, OR (US)

(21) Appl. No.: 18/613,292

(22) Filed: Mar. 22, 2024

Related U.S. Application Data
(60) Provisional application No. 63/491,809, filed on Mar. 23, 2023.

(51) Int. Cl. C12N 1/16; A01N 63/32; A01P 3/00; C12R 1/645

(52) U.S. Cl. CPC C12N 1/165; A01N 63/32; A01P 3/00; C12R 2001/645

(57) ABSTRACT

The present disclosure provides a novel biofungicide produced by Coniochaeta velutina by growing the fungus in artificial media and collecting the supernatant, as well as methods of using the biofungicide. This organic biofungicide shows various characteristics making it suitable for application to crops to treat or prevent fungal diseases, such as early blight of potato.

### CROSS-REFERENCE

The present application claims priority to U.S. Provisional Patent Application Ser. No. 63/491,809 filed Mar. 23, 2023, the contents of which are expressly incorporated herein by reference.

### BACKGROUND OF THE INVENTION

### Field of Invention

The present disclosure provides a novel biofungicide produced by Coniochaeta velutina by growing the fungus in artificial media and collecting the supernatant, as well as methods of using the biofungicide. This organic biofungicide shows various characteristics making it suitable for application to crops to treat or prevent fungal diseases, such as early blight of potato.

### Background

Management of plant diseases through use of natural products currently relies on a limited number of microbial sources. For plant disease management, use of beneficial bacteria, principally Bacillus and Pseudomonas species, generally requires viable cells that produce antimicrobial chemicals and lytic enzymes after application. The same is true for beneficial fungi, where antimicrobial production occurs after the spores of fungi such as Trichoderma species germinate. The need for viable cells can make formulations more difficult, and limit options for tank-mix applications where they may be combined with other agrichemicals at reduced chemical rates (Carvalho et al., Biocontrol Sci. Tech., (2022), 32:10, 1220-31).

Another type of microbial product for plant disease management comes from microbes capable of producing antimicrobial compounds that can be produced in culture, extracted, and applied in a manner more like conventional agrichemicals. One of the most successful discoveries in this area was identification of strobilurins from a soil-dwelling, mushroom-forming basidiomycete Strobilurus tennicellus (Anke, T., Can. J. Botany, (1995), 73: S1, 940-5; Bartlett et al., Pest Management Sci., (2002), 58, 649-662). The original compound required chemical modification to become photostable, however the product originated from a natural product, and remains one of the most widely used products in conventional agricultural production.

Additional sources of natural products are available in endophytic fungi that colonize different plant tissues. The beneficial properties of some plants used in herbal medicine have been attributed to the endophytic fungus within the plant (Yan et al., Appl. Microbiol. Biotech., (2019), 103, 3327-40). In some cases, it is proposed that the endophyte protects the plant through production of antimicrobial compounds. While this has been proposed, thus far there are very few examples where fermentation of endophytic fungi resulted in filtrates useful in plant disease management (Terkar & Borde, “Endophytic fungi: Novel source of bioactive fungal metabolites” in New and Future Developments in Microbial Biotechnology and Bioengineering: Recent Advances in Application of Fungi and Fungal Metabolites: Current Aspects, pg. 95-105, (2020) ed. Singh, J. & Gehlot, P.).

There are very few examples of natural antifungal products. Commercial products for foliar application rely primarily on Bacillus subtilus and Bacillus amyloliquefaciens strains. In each case the bacterial cells are present in the applied product. Trichoderma and Chaetomium fungal species have also been used as antifungal agents, primarily for soil incorporation, and rely on living spores that germinate. In the case of both Chaetomium and Trichoderma (Gliocladium), there are a combination of factors involved in biocontrol activity. Enzymes such as chitinases and glucanases are produced (van Tilburg and Thomas, Appl. Environ. Microbiol., (1993) 59:236-42), along with antifungal compounds such as gliotoxin (Jones & Hancock, Microbiol., (1988) 134: 2067-75; Howell et al., Biocontrol Sci. Tech., (1993) 3:435-41) and chetomin (Pietro A., Phytopathol. (1993) 82:131-35). Surprisingly, the two most effective fungi used to control other fungi, Chaetomium and Gliocladium, both rely on production of epipolythiodioxopiperazines, sulfur bridge containing molecules (Waring & Beaver, Gen. Pharmacol. (1996) 27:1311-16). Potential phytotoxicity of compounds such as chetomin, gliotoxin and viridiol (Jones & Hancock, Can. J. Microbiol., (1987) 33:963-966) have limited the direct application of culture filtrates to target foliar pathogens. Application of cell-free Coniochaeta culture filtrates provides a new avenue for specific foliar plant pathogen control.

During a study on the endophytic fungi present in the walking iris plant (Neomarica gracilis) two isolate types of the fungus Coniochaeta were identified. Unlike many other studies on endophytic fungi, where multiple fungal genera are found in the study plant, we found only the two Coniochaeta isolates. One isolate had a strong inhibitory action against the other isolate, as well as certain other fungi, and herein we detail the production and use of a new natural product for plant disease management.

### SUMMARY OF THE INVENTION

The present disclosure provides, in part, a supernatant of an artificial liquid culture of Coniochaeta velutina strain NRRL 68077. In some embodiments, the supernatant is filtered, and may be substantially cell-free. In some embodiments, the artificial liquid culture is potato dextrose broth. In some embodiments, the supernatant is not treated with an organic solvent. In some embodiments, the supernatant was collected from a culture at least 2-weeks old, or at least 3-weeks old.

The present disclosure further provides a water-soluble fraction of a supernatant of an artificial liquid culture of Coniochaeta velutina strain NRRL 68077. The present disclosure further provides a water-soluble fraction of a supernatant of an artificial liquid culture of Coniochaeta velutina, wherein the water-soluble fraction comprises fungicidal activity against Alternaria solani, and wherein the non-water-soluble fraction does not comprise fungicidal activity against Alternaria solani. In some embodiments, the C. velutina is strain NRRL 68077. In some embodiments, the water-soluble fraction has not been contacted with methanol.

The present disclosure further provides a living plant and any of the supernatants or supernatant fractions described herein.

The present disclosure further provides a method of killing a fungal cell, comprising contacting an effective amount of any of the supernatants or supernatant fractions described herein to the fungal cell. In some embodiments, the supernatant is diluted by at least 1:10. In specific embodiments, the fungal cell is of the species Alternaria solani.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1A, FIG. 1B, FIG. 1C, and FIG. 1D provide graphical and photographic representation of Coniochaeta velutina biomass production in three liquid media. Replicate flasks with 100 ml of medium were inoculated with a spore solution of Coniochaeta and shake incubated (100 rpm) at 20° C. for up to three weeks. Three individual flasks were chosen for each media at one-week intervals. Mycelium was harvested by vacuum filtration on pre-weighed Whatman paper and dried at 37° C. Filtrates were pooled for three flasks and used for bioassay. FIG. 1A) Weekly dry weight. FIG. 1B) Cultures after one week on potato dextrose broth (left), malt extract (center) and malt extract broth (right). FIG. 1C) two-week-old cultures FIG. 1D) three-week old cultures. Cultures grown in potato dextrose broth and malt extract became brown within two weeks while cultures in malt extract broth (malt extract plus peptone) remained pale yellow to orange.

FIG. 2 provides photographic representation of inhibitory (I) and non-inhibitory (N-I) isolates of C. velutina isolated from walking iris. Inhibition of Alternaria solani (As) growth on potato dextrose agar is apparent for one of the two isolates (NRRL 68077).

FIG. 3 provides photographic representation of streak cultures of the bacterium Bacillus amyloliquefaciens (Ba) (STARGUS isolate) and the ascomycete yeast Moesziomyces aphidis (Ma). Wells contained 100 ul of C. velutina culture filtrate. After incubation for 72 hr, no inhibition zones were seen.

FIG. 4A and FIG. 4B provide photographic representations of the inhibition of Trichoderma asperellum mycelial growth on potato dextrose agar. Colonies of C. velutina (Co) and B. amyloliquefaciens (Ba) were grown for one week, followed by inoculation in the center with a mycelial plug of T. asperellum (Ta). FIG. 4A) Four days after inoculation with T. asperellum. FIG. 4B) seven days after inoculation with T. asperellum. C. velutina is overgrown while B. amyloliquefaciens maintains an inhibition zone.

FIG. 5 provides graphical representation of data showing reduction in A. solani infection sites after treatment with C. velutina culture filtrate. Terminal trifoliates from Kennebec potato plants were sprayed to runoff with spreader/sticker alone (control), three-week old fungal filtrate (1:20 dilution of filtrate) or StarGus bacterial biofungicide (1:100 dilution), incubated for 12 hours, then spray-inoculated with A. solani spores. Individual infection sites were counted for each leaf at four days. Box and whisker plot with average deviation bars show a similar overall reduction in successful infections when leaves are treated with Coniochaeta filtrate or STARGUS.

FIG. 6 provides graphical representation of data showing reduction in A. solani lesion size after treatment with C. velutina culture filtrate. Terminal trifoliates from Kennebec potato plants were sprayed to runoff with spreader/sticker alone (control), three-week old fungal filtrate (1:20 dilution of filtrate) or STARGUS bacterial biofungicide (1:100 dilution), incubated for 12 hours, then spot inoculated with 100 ul of spore suspension at three spots on each leaf. Lesion measurements were made 6 days later. Box and whisker plot with average deviation bars show a similar overall reduction in lesion sizes when leaves are pretreated with filtrate (1:20) or STARGUS (1:100).

FIG. 7 provides graphical representation of data showing inhibition of A. solani hyphal growth on solid medium by C. velutina filtrate. Hyphal plugs were placed on potato dextrose agar plates 20 mm from wells containing 100 ul of three-week-old liquid culture filtrate. Measurements were made beginning at three days and continued until growth to control treatments were reached. Hyphae were unable to grow after four days, demonstrating a similar activity with liquid production of antifungal activity and solid medium production.

FIG. 8 provides photographs of BioGel P-2 columns, flow through, and four water washes. For each, 21 ml of culture filtrate was added to the column and for each wash, 21 ml water was added. Flow through is shown on the left, followed sequentially by water washes 1-4.

FIG. 9 provides photomicrographs of A. solani treated with C. velutina culture filtrate. A. solani spores were exposed immediately upon incubation to 1:40 and 1:20 dilutions of culture filtrates. Germ tube development and melanin production, both required for plant infection, are prevented at 48 hours when compared to the control.

FIG. 10 provides photomicrographs of A. solani treated with C. velutina culture filtrate. A. solani spores were exposed 24 hours after plating to 1:40 and 1:20 dilutions of culture filtrates and incubated for an additional 24 hours in the presence of culture filtrate Germ tube development and melanin production, both required for plant infection, are slowed or lowered when compared to the control.

### STATEMENT OF DEPOSIT

Strains representative of the inventions disclosed herein were deposited on Oct. 28, 2021, under the terms of the Budapest Treaty with the Agricultural Research Service (ARS) Patent Culture Collection. A representative Coniochaeta velutina strain was deposited under ARS Patent Culture Collection Reference No. NRRL 68077. The microorganism deposits were made under the provisions of the “Budapest Treaty on the International Recognition of the Deposit of Microorganisms for the Purposes of Patent Procedure”. All restrictions on the availability to the public of these deposited microorganisms will be irrevocably removed upon issuance of a United States patent based on this application. For the purposes of this invention, any C. verulina strains having the identifying characteristics of NRRL 68077, including subcultures and variants thereof which have the identifying characteristics and activity as described herein are included.

### DETAILED DESCRIPTION OF THE INVENTION

An isolate of the fungal genus Coniochaeta velutina (NRRL 68077) was found growing as an endophyte in leaves of walking iris (Neomarica gracilis). Initial studies on solid media revealed a strong zone of inhibitory activity. Cultures were grown in both synthetic and non-defined liquid media. Growth was poor on synthetic media but was readily supported on potato dextrose broth, malt extract and malt extract broth. Optimal growth and antifungal activity was found after growth in shake culture for one to two weeks in potato dextrose broth. Filtrates of the culture media were fully inhibitory to Alternaria solani and Alternaria brassicicola spore germination at dilutions of, for example, 1:20. Filtrates also inhibited mycelial growth of these fungi on solid media. Limited inhibition was found for Sclerotinia sclerotiorum and Sclerotium rolfsii, and no inhibition of Trichoderma asperellum, Esherichia coli or Bacillus amyloliquifaciens was noted. Application to potato leaves followed by inoculation with Alternaria solani resulted in delayed and reduced lesion development at a level similar to a bacterial biofungicide application. The active component of the filtrate is highly water-soluble, non-enzymatic and not effected by pH levels. The active component is not extractable with organic solvents. Simple production systems, targeted inhibitory activity, and chemical stability suggest the diluted filtrates may be a useful new source of biofungicide.

The role of endophytes in protection of plants from disease has been suggested, with the primary role being attributed to enhanced activation of the plant defense system (Latz et al., Plant Ecol. Diversity, (2018) 11:555-67; Fontana et al., Pathogens, (2021) 10:570). Antibiotic production by an endophyte may be limited while inside the plant where there is limited competition for resources due to a limited number of endophytes. Only a small portion of plant tissue is generally colonized by an endophytic fungus. Activation of antibiotic production may be more useful after plant senescence. In the case of Coniochaeta growing endophytically in a monocot, the fungus may be producing the highly water-soluble antifungal compounds, which could easily be translocated through the leaf veins. Growth in culture allows for amplification of production beyond what would be seen in the nutrient limited extracellular regions of a plant leaf.

The genus Coniochaeta encompasses an expanding group of species. Lifestyles range from saprophytes to endophytes. Some have highly effective enzymes and have been useful in biofuel production (Nichols et al., “Use of Coniochaeta ligniaria to detoxify fermentation inhibitors present in cellulosic sugar streams.” In: Hou, C. T., Shaw, J.-F., editors. Biocatalysis and Molecular Engineering. New York: John Wiley and Sons. p. 253-263 (2010)). The Coniochaeta velutina isolate described herein lacks strong cellulase and xylanase activity but may harbor other enzymes that aid in its endophytic lifestyle and is the first Coniochaeta species reported to be isolated from the monocot walking iris. The genus Coniochaeta contains many newly identified species (Arnold et al., Int'l J. Systematic Evol. And Microbiol., (2021) 71:11) and it is likely that a species will have differences at the isolate level when being evaluated for enzyme and antibiotic activity. Trichoderma (Gliocladium) virens is an example where isolates varied in the antibiotics they produced (Howell & Puckhaber, Biol. Control, (2005) 33:217-22), as well as Chaetomium (Linkies et al., J. Appl. Microbiol., (2020) 131:375-91).

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

An “effective amount” is an amount sufficient to effect desired beneficial or deleterious results to a target organism. In terms of treatment, an “effective amount” is that amount sufficient to make the target microbe (e.g., fungus or bacterium) non-functional by causing an adverse effect on that microbe, including (but not limited to) physiological damage to the microbe; inhibition or modulation of growth; inhibition or modulation of reproduction; inhibition of morphological transition; or increased mortality compared to untreated microbes. The exact amount of C. velutina-derived biofungicide required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The term “artificial media” and grammatical variants thereof means any composed fungal growth medium capable of supporting growth of C. velutina. Such media include, for example, potato dextrose broth, where components of the media are derived from natural sources, but are not the natural source themselves.

The term “artificial culture” as used herein, refers to fungal cultures grown in vitro.

The embodiments illustratively disclosed herein suitably may be practiced in the absence of any element [e.g., method (or process) steps or composition components)] which is not specifically disclosed herein. Thus, the specification includes disclosure by silence. Written support for a negative limitation may also be found through the absence of the excluded element in the specification, known as disclosure by silence.

### Fungal Cultures

One of skill in the art will recognize that multiple culture conditions can be modified in practicing the inventions disclosed herein. Non-limiting examples of culture conditions that can be modified during the application and practice of the inventions disclosed herein, include temperature, media carbon sources, media nitrogen sources, oxygen concentration, pH, predominant morphological form, age/growth phase of a culture, and set up and organization of an industrial fermenter. One of skill in the art will recognize that other culture parameters affecting desired production parameters and yield can be modified.

In one aspect of the invention, cultures of C. velutina described herein can be grown at any temperature that facilitates the production of one or more biofungicide. For example, a culture can be grown at a temperature of 15°−30° C., or any whole or partial degree within that range, including, but not limited to 15.0° C., 15.5° C., 16.0° C., 16.5° C., 17.0° C., 17.5° C., 18.0° C., 18.5° C., 19.0° C., 19.5° C., 20.0° C., 20.5° C., 21.0° C., 21.5° C., 22.0° C., 22.5° C., 23.0° C., 23.5° C., 24.0° C., 24.5° C., 25.0° C., 25.5° C., 26.0° C., 26.5° C., 27.0° C., 27.5° C., 28.0° C., 28.5° C., 29.0° C., 29.5° C., and 30.0° C.

In some embodiments, C. velutina, including specific strains described herein, can be grown under conditions where the pH of the culture facilitates the production of one or more bioproducts of interest. For example, a culture can be grown in media where the pH is between 4.5 and 8.5, 6.0 and 7.5, or any value within that range, including, but not limited to pH 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5. One of skill in the art will recognize that a stable pH does not need to be maintained throughout the entirety of the growth of the strain producing the bioproduct(s) of interest. Thus, in some embodiments, the pH of a microbial culture of the present invention will vary. In other embodiments, pH buffers can be added to maintain a relatively stable pH where the pH of the culture medium over the life of the culture does not vary from a chosen starting point by more than ±0.5.

In some embodiments, C. velutina as utilized in the instant disclosure can be grown in the presence of particular carbon sources. For example, a culture can be grown in the presence of simple carbon sources such as arabitol, sucrose, fructose, glucose, mannose, galactose, arabinose, arabinose, xylose, mannitol, glucitol, galactitol, xylitol, ribitol, threitol, glycerol, gluconic acid, glucosamine, or meso-erythritol. Alternately, a culture can be grown in the presence of complex carbon sources such as cellulose, starch, beet molasses, carob pod, cornmeal hydrolysates, corn syrup, fuel ethanol fermentation stillage, grape skin pulp, vegetable oils, peat hydrolysate, hydrolyzed potato starch, and spent sulfite liquor. Media for use in the present invention can include amino acids such as aspartate, threonine, lysine, methionine, isoleucine, asparagine, glutamic acid, glutamine, proline, alanine, valine, leucine, tryptophan, tyrosine, phenylalanine, and their metabolic intermediates. Other media useful in the instant disclosure include those well known in the art, such as potato dextrose medium, Saboraud dextrose medium, malt broth, malt extract, and brain heart infusion medium. These lists are non-limiting and it is well within the capabilities of one of skill in the art to utilize other carbon sources or specific media in practicing the present invention.

Other nutritional parameters can also be varied, including nitrogen sources. Non-limiting examples of nitrogen sources include organic nitrogen sources (e.g., peptone, yeast extract, malt extract, and soybean flour) and inorganic nitrogen sources (e.g, urea, ammonium sulfate, ammonium chloride, ammonium phosphate, ammonium carbonate and ammonium nitrate) can be included in growth media. Phosphate sources such as potassium dihydrogen phosphate, dipotassium hydrogen phosphate and their corresponding sodium-containing salts can be included in growth media as necessary. Metal and mineral salts such as salts of zinc, iron, magnesium, manganese, calcium and copper can be included as needed. Other nutritional supplements, such as vitamins (e.g, biotin, thiamine) can also be included. One of skill in the art will recognize that varying culture nutritional makeup can be utilized to maximize production of a biofungicide of interest. Any of these nutrients can be used alone or in combination with any other nutrient.

Nutrients can be added to the culture in any feeding regimen, including, but not limited to high cell-density culture, batch culture, fed-batch culture, constantly-fed-batch culture, exponentially fed batch culture, continuous culture, or a mixture of these approaches for different nutrients.

In some instances, the length of time a culture is grown can be modified to enhance or begin production of a biofungicide of the instant disclosure. For example, a culture can be grown for 10-300 hours, or more, or any time point within that range, for example 10, 20, 30, 40, 50, 60, 70, 80, 90, 100, 110, 120, 130, 140, 150, 160, 170, 180, 190, 200, 210, 220, 230, 240, 250, 260, 270, 280, 290, 300 hours, or more before harvesting of a bioproduct commences. Culture growth time can also be measured in days, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more days. Culture growth time can also be measured in weeks, for example, 1, 2, 3, 4, 5, or more weeks.

Additionally, optimization of biofungicide production by C. velutina may depend on growing a culture to a particular point in the life cycle. For example, a culture can be grown to early lag phase, middle lag phase, late lag phase, early exponential phase, mid-exponential phase, late exponential phase, early stationary phase, mid-stationary phase, or death phase. In some instances, cultures can be maintained in a growth phase (e.g., by fed-batch culture) to maintain a particular growth phase for the culture.

### Supernatant Collection

After a suitable time for culturing, which can be determined by one of skill in the art, the cell-free portion can be collected from the culture. Cultures useful in practicing the instant disclosure can be any liquid tissue culture comprising C. velutina (e.g., strain NRRL 68077), for example, submerged or floating culture. The portions of the culture to use with the present invention includes cell-free parts or portions of the culture, culture supernatant or filtrate, or any proportions or fractions thereof. In preferred embodiments, a filtrate or supernatant of the instant disclosure containing a biofungicide is not treated with an organic solvent, such as methanol, chloroform or dichloromethane. Preferably, the portion of the culture utilized is the cell culture supernatant or cell culture filtrate. Filtrates and supernatants of the instant invention are preferably cell-free.

Cell-free portions of the culture filtrate or supernatant can be prepared by any method known in the art to separate cell culture supernatant fluids. For example, the culture may be filtered by any means known in the art to obtain the filtrate, such as, for example, 0.2 μm filters and the like. Alternatively, the cell-free portion of the culture may be collected by centrifugation.

Optionally, the liquid tissue culture can be treated to reduce or eliminate the viability of live organisms, such as pasteurization or sterilization, by methods known in the art. The collected liquid tissue culture may be pasteurized or sterilized either before or after separation to obtain the cell-free portion of the culture, by any method known in the art. The filtrate or supernatant may have its volume or liquid component adjusted as determined by one of skill in the art to produce concentrates, diluates, or dried powders. Drying can be performed via any method known in the art, including the use of open-air drying, small batch desiccators, vacuuform dryers, fluid beds or spray dryers, or freeze-driers.

### Application of Biofungicides

Compositions disclosed herein can be applied to soil, fruits, vegetables, crops, and any other desired target using any delivery methodology known to those of skill in the art. For example, biofungicide compositions can be applied to the desired locale via methods and forms including, but not limited to, sprays, baits, granules, flood/furrow methods, sprinklers, fumigation, root soaking and drip irrigation. In embodiments of the invention where the compositions are sprayed onto a desired locale, the compositions can be delivered as a liquid suspension, emulsion, microemulsion or powder. In other embodiments, granules or microcapsules can be used to deliver the compositions of the invention.

The compositions of the present invention can be applied to plants and/or crops by any convenient method, for example, by using a fixed application system such as a center pivot irrigation system. Application to fields of plants and/or crops is made by air spraying, i.e., from an airplane or helicopter, or by land spraying. For example, land spraying may be carried out by using a high flotation applicator equipped with a boom, by a back-pack sprayer or by nurse trucks or tanks. One of skill in the art will recognize that these application methodologies are provided by way of example and that any applicable methods known in the art or developed in the future can be utilized.

Where desired, biofungicides of the instant disclosure can be combined with any additional treatment or crop application, including but not limited to, fertilizers, pesticides, insecticides, and fungicides. Such combinations, and the appropriate formulations and method of application can readily be determined by the skilled artisan.

Having generally described this invention, the same will be better understood by reference to certain specific examples, which are included herein to further illustrate the invention and are not intended to limit the scope of the invention as defined by the claims.

### EXAMPLES

### Example 1

Isolation and Initial Characterization of C. velutina Strains

Leaf samples from walking iris, maintained as a houseplant for over twenty years, were cut laterally in approximately 10 cm strips, immersed in 70% ethanol for 1 min, transferred to 8% sodium hypochlorite for three min, then rinsed in sterile distilled water for one min. Samples were blotted on sterile paper towels and plated on potato dextrose agar plates. Plates were maintained at room temperature for two weeks. Two colony types appeared, a pale orange and a darker brown type. The darker colony produced an inhibition zone towards the pale orange colonies. Colonies were transferred to individual plates and identified by ribosomal sequences.

The inhibitory isolate (NRRL 68077) was cultured in potato dextrose broth, then hyphae were transferred to bead beater tubes and disrupted by two 20 sec cycles. Samples were then processed for DNA isolation using a Qiagen plant DNA mini kit according to manufacturer instructions. Final DNA samples were eluted in 50 microliters of water.

Fungal ribosomal small subunit ITS regions 1 to 4 were amplified in reaction mix containing 2 microliters DNA, 1 microliter each of two ITS-specific primers, sixteen microliters of water and twenty microliters of MyTaq Plant PCR mix (Bioline). Amplification was performed by 3 min at 95° C., followed by 30 cycles of 52° C. for 15 sec and 72° C. for 1 min, ending with five min at 72° C. A second amplification was performed using 1 microliter of the first reaction, 1 microliter each two different ITS-specific primers, seventeen microliters water and twenty microliters of MyTaq Plant PCR mix. The same PCR cycle as the first amplification was used. The final product was sequenced (Macrogen) and compared to known sequences using NCBI BLASTn.

There are no previous reports on endophytic fungi from walking iris, so it was unexpected to find two different Coniochaeta isolates, and to find them in a monocot. Colonies were slow growing, orange to brown, producing very small phialospores. Sequence from the ITS1-4 region indicated a species related to Coniochaeta velutina. The culture was deposited at the USDA-ARS fungal collection in Peoria, IL as NRRL 68077.

### Example 2

### Production of Biofungicide

Cultures of C. velutina (NRRL 68077) were maintained on potato dextrose agar plates. Confluent cultures were prepared by flooding an individual plate with water and transferring the conidial suspension to new plates, followed by growth for two weeks at room temperature. Liquid cultures were prepared in 250 ml flasks, adding 100 ml of potato dextrose broth (Difco #254920), malt extract (Sigma-Aldrich #70167) or malt extract broth (Sigma-Aldrich #70146), followed by autoclave sterilization. Individual flasks were inoculated using 1 ml of conidial suspension generated by flooding three plates each with 10 ml sterile water and pooling the conidial suspensions. Inoculated flasks were placed in a shaking incubator (100 rpm) at 20° C. Replicate flasks were harvested at one, two and three weeks after inoculation. Mycelium was collected by vacuum filtration, dried at 37° C. on pre-weighed filter and measured as dry weight. To ensure that conidia were not included in the filtrate toxicity studies, filtrates were passed through a Nalgene Rapid Flow filter and maintained in 150 ml sterile flasks.

To test for inhibitory action, filtrates were diluted at 1:1, 1:5, 1:10, 1:15, 1:20 and 1:40 by adding appropriate filtrate volumes to potato dextrose broth in 24-well polystyrene plates (Corning), resulting in one ml total volume. Inhibition was assessed microscopically after two days. Fungi tested were Alternaria solani, A. brassicicola, Botrytis cinerea, Monolinia fructicola, Penicillium digitatum, Phytophthora infestans, Sclerotium rolfsii, Sclerotinia sclerotiorum and Trichoderma asperellum. Inoculation of individual wells was performed using 50 ul conidial suspension (approx. 1,000 spores), and hyphal disks (5 mm) from non-sporulating test isolates (S. rolfsii and S. sclerotiorum). Effective dilutions were scored based on elimination of growth or germination.

Filtrates were size fractionated using Centriplus concentrators (Amicon) with a 10 kDa cutoff and a 3 kDa cutoff. Samples were centrifuged according to manufacturer protocols. Samples from the retentate and flow through were tested at 1:10, 1:20 and 1:30 dilution in potato dextrose broth using A. solani spores as previously described.

Filtrates were passed over a C18 reversed phase silica gel (Sigma) contained in a 2.5×10 cm Flex column (Kimble). One volume of water was passed through the hydrated gel, followed by the filtrate. Another volume of water was passed through, followed by one volume of absolute ethanol. Each fraction was tested in a liquid bioassay with Alternaria solani spores as previously described.

Culture filtrates were also extracted with chloroform or dichloromethane. Five hundred microliters of filtrate was extracted with five hundred microliters of organic solvents placed in a 1.5 ml microfuge tube. Samples were briefly shaken, centrifuged for one minute at 12K rpm and the supernatant (filtrate) removed by pipetting. Extracted samples were placed in an Eppendorf vacufuge for 20 min to remove any residual organic solvents. Samples were tested for activity in a liquid bioassay with Alternaria spores as previously described.

Filtrate was also tested against Alternaria solani hyphal growth by adding 100 ul of a 1:20 dilution of three-week old potato dextrose broth filtrate to 8 mm wells cut in plates of potato dextrose agar. Plates were inoculated with mycelial plugs of A. solani and inhibition of mycelial growth measured beginning at three days after inoculation.

Testing of single cell microbes was performed on solid medium. Filtrates were added to individual plate wells at each dilution followed by streaking cultures of the bacteria Esherichia coli and Bacillus amyloliquefaciens. The streak test was also used to test inhibition of the plant associated ascomycete yeast Moesziomyces aphidis. Plates were incubated at 37° C. for 30 hr (E. coli) or room temperature for 72 hr (B. amyloliquefaciens and M. aphidis).

### Results

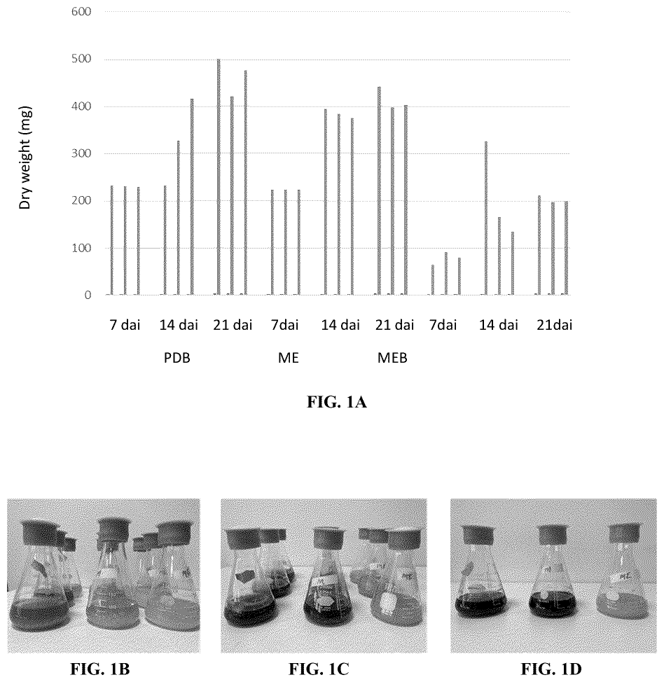

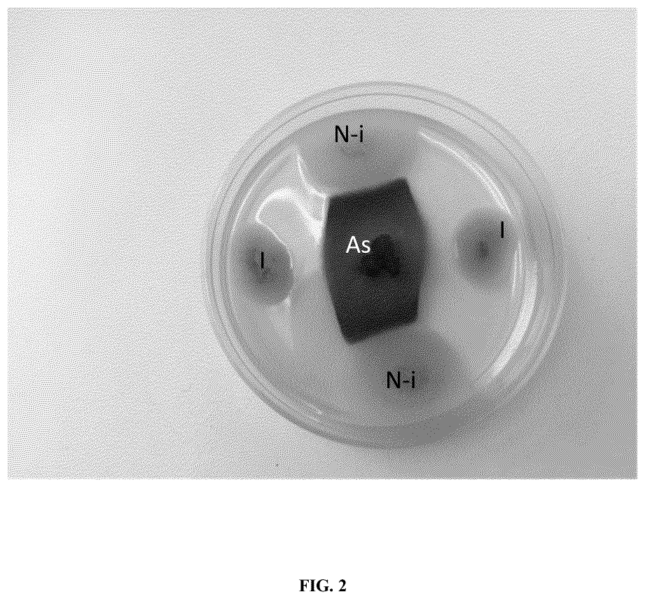

Initial attempts to culture Coniochaeta on synthetic medium with defined nitrogen and carbohydrate sources resulted in very poor growth. Culturing on less-defined media resulted in suitable levels of growth so further studies involved inexpensive media sources which included potato dextrose broth, malt broth and malt extract broth (malt extract plus peptone). Optimal growth was seen with potato dextrose broth and malt extract (FIG. 1) while malt extract broth growth resulted in viscous masses of hyphae that made filtering difficult, with ineffective inhibitory activity in the filtrates. This suggests that C. velutina (NRRL 68077) may be adapted to require some component that is available in plant derived media (potato or sprouting grain) and that nitrogen levels influence production of inhibitory components of the filtrate. All further studies used potato dextrose broth. Full inhibitory activity was seen within two weeks of shake cultivation, while the inhibitory activity remained stable at three weeks of cultivation, indicating no detoxification of the inhibitory activity. Variations in growth between replicates of the same medium at a given time point were common, and likely represent amounts of inoculum initially present, therefore all testing was done with pooled three-week old culture filtrates to minimize variation in final filtrates. It is important to note that the inhibitory nature of C. velutina (NRRL 68077) was seen in both solid media and liquid media (FIG. 2, inhibitory strain).

Inhibitory activity is likely due to a non-enzymatic component of the filtrate as the fractions below 10 kDa and 3 kDa retained full activity. Column chromatography on silica gel indicated that activity is in the highly polar, water-soluble fraction, as the full activity came through the first pass of the filtrate through a silica gel column.

Filtrate samples extracted with the organic solvents chloroform and dichloromethane retained their activity, indicating that the inhibitory activity was not present in the non-polar, organic hydrocarbon fraction.

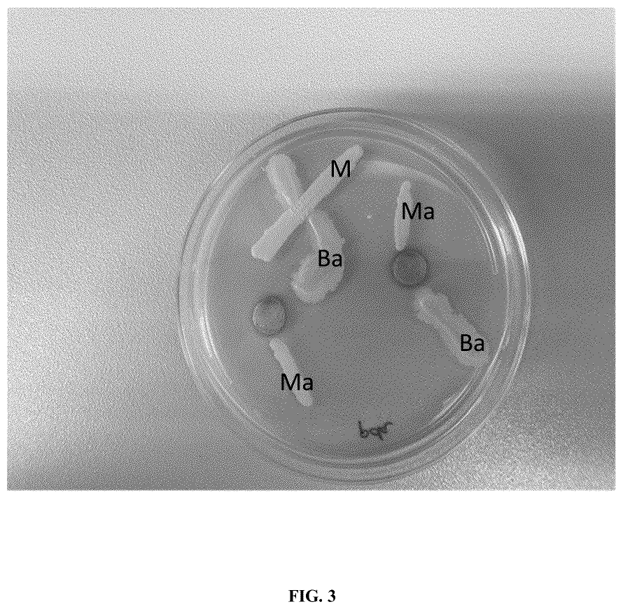

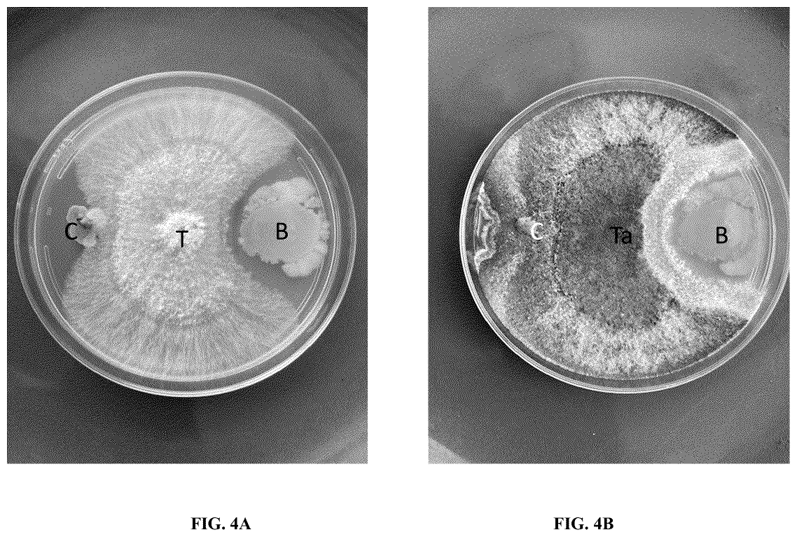

The filtrate from three-week old potato dextrose broth cultures of C. velutina showed a defined spectrum of activity (Table 1). For these studies, filtrates were diluted at 1:1, 1:5, 1:10, 1:15, 1:20 and 1:40 by adding appropriate filtrate volumes to potato dextrose broth in 24 well polystyrene plates, resulting in one ml total volume. Inhibition was assessed microscopically after two days. Alternaria species were the most sensitive, while the ascomycete Sclerotinia sclerotiorum and the basidiomycete Sclerotium rolfsii were only inhibited slightly at very high filtrate levels. A fungus commonly used as a biological control, Trichoderma asperellum was not inhibited in liquid culture or solid medium culture (FIG. 4A, FIG. 4B), nor was the gram-negative bacterium E. coli or the gram-positive bacterium B. amyloliquefaciens. The ascomycete yeast M. aphidis was also not inhibited (FIG. 3).

TABLE 1
Inhibition by C. velutina (NRRL 68077)

| Fungal isolate | Inhibitory dilution level of filtrate |
|---|---|
| Alternaria solani | 1:20 to 1:40 |
| Alternaria brassicicola | 1:20 to 1:40 |
| Monolinia fructicola | 1:15 to 1:20 |
| Botrytis cinerea | 1:05 |
| Sclerotinia sclerotiorum | 1:05 |
| Sclerotium rolfsii | 1:05 |
| Phytophthora infestans | 1:05 |
| Trichoderma asperellum | No inhibition |
| Penicillium digitatum | No inhibition |

### Example 3

Protection of potato from A. solani early blight with C. velutina biofungicide

Plant inoculations were performed on Kennebec potato leaflets obtained from the lower half of 50 to 60-day old greenhouse-grown plants. For control treatments, terminal trifoliate leaflets were sprayed to runoff with water solutions containing Turbo spreader sticker (Bonide) at 4 microliters per ml. Biofungicide treatments consisted of the same sticker solution plus either filtrate from three-week-old potato dextrose broth shake cultures of C. velutina (NRRL 68077) (1:20 dilution) or a 1:100 dilution of Bacillus amyloliquefaciens strain F727 (STARGUS, Marrone Bioinnovations). Replicate leaflets were incubated for twelve hours in shallow glass trays covered with clear plastic before inoculation with A. solani.

Spores were collected from confluent potato dextrose agar cultures of A. solani grown for three weeks under lighting from UVb fluorescent bulbs (Zilla Slimline). Spores harvested by flooding plates with sterile water and dislodging with a glass rod were collected for counting with a haemocytometer. Spore concentrations were adjusted to 1×10⁴ spores per ml.

Leaflets were subsequently inoculated with Alternaria solani spores using two methods. Spot inoculations were made with three aliquots (100 ul) per leaf. Lesion measurements were made after 6 days incubation in the covered glass plates. Spray inoculations were made by spraying a spore suspension onto each leaf and counting lesion sites after 4 days.

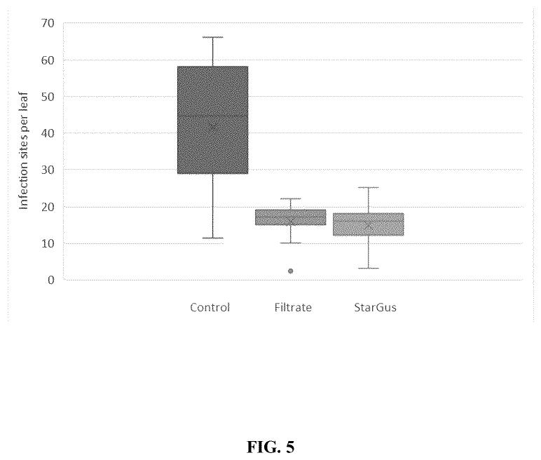

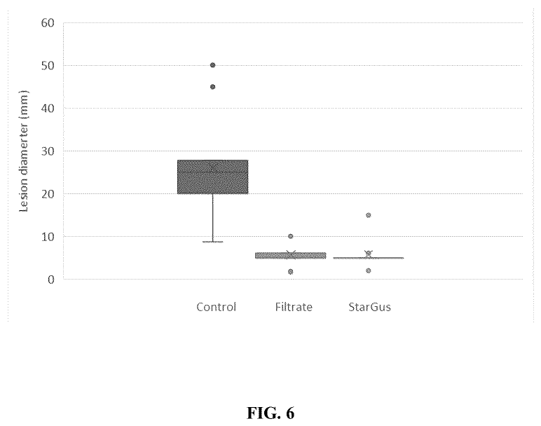

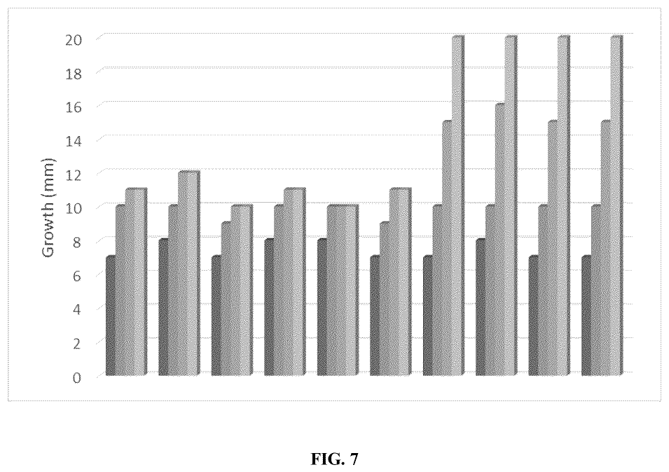

Spray treatments using either C. velutina (NRRL 68077) filtrate (1:20 dilution) or B. amyloliquefaciens (STARGUS) provided high levels of protection from infection of potato leaves by A. solani (FIG. 5 and FIG. 6). Spot inoculations represent a more concentrated spore level at one site and multiple infections would result in larger, focused lesions. Spray inoculations represent lower spore concentrations at a given site of infection. Both treatments resulted in lower degrees of infection. Once established, Alternaria is less likely to be inhibited as neither treatment has systemic properties. In addition to inhibition of spore germination, the filtrate also inhibits growing hyphae of A. solani (FIG. 7), which would result in a further delay of infection as hyphae grow from the germinating spore before initiating infections.

Characteristics of the C. velutina (NRRL 68077) filtrates make them highly suitable for use in both organic and conventional agricultural production. Production occurs on simple potato dextrose liquid media and can be used directly. Cultures of C. velutina (NRRL 68077) grew poorly on synthetic liquid media with defined carbon and nitrogen sources but grew very well on naturally derived substrates such as potato broth or malt extract. The addition of peptone to malt extract resulted in an unsuitable type of flocculant growth and reduced antifungal activity. There is no need for viable cells in the applied material, thus a mixture of low-rate fungicide and fungal filtrate would be possible in conventional production systems, while filtrate alone would be suitable in organic systems. It does not matter if Coniochaeta cells are present in treatments as the spores are very small and should not interfere with spray nozzles.

While the effective inhibitory range is limited to some fungi, this is beneficial from the basis that it does not cause indiscriminate inhibition of fungi and bacteria. Trichoderma (Gliocladium) is one of the most common fungi used as a biocontrol agent for fungal diseases, and it is not inhibited by the filtrate. The C. velutina (NRRL 68077) filtrate lacks general antibacterial activity, which is beneficial if being included in treatments with bacterial biocontrol agents, especially for Bacillus species used in pathogen and insect management. It also lacks inhibitory activity towards at least one yeast, and phylloplane yeast can play a positive role in limiting foliar pathogen infections (Kitamoto, H., FEMS Yeast Res., (2019) 19:foz053).

The activity of the C. velutina (NRRL 68077) filtrate described herein is protective in nature when applied to leaf surfaces and is highly water soluble. While it is possible there may be further benefits for disease control if Coniochaeta spores were included, the slow growth relative to microbes such as Trichoderma make use of a spore inoculum less likely to be effective on its own.

There have been two reports of antimicrobial production by Coniochaeta species. The first example is coniocetin, produced by the dung saprophyte C. ellipsoides (Segeth et al., J. Antibiotics, (2003) 56:114-22). Interestingly, the inhibitory compound (s) is only produced on solid substrate fermentation (SSF) and is soluble in non-polar solvents, both features not found with the strain reported in our study. The spectrum of inhibition also differs from our filtrate in that coniocetin inhibits yeast and bacteria.

The second example comes from cultures of C. velutina strain PC27-5, which was found as an endophyte of Western hemlock (Xie et al., J. Microbiol., (2015) 53:390-97). The strain produces antifungal compounds on both solid and liquid medium. The spectrum of inhibitory activity is different from the strain and filtrates described above in that we found little effect against Sclerotinia sclerotiorum or Phytophthora infestans, and the filtrate was not fungicidal towards Phytophthora, as the hyphae initiated growth after transfer to from filtrate-containing liquid medium to potato dextrose agar plates. The activity of strain PC27-5 was attributed to anthroquinone glycoside production, and extraction was performed using the organic solvent dichloromethane, however filtrate activity of our strain is not extractable with organic solvents.

### Example 4

Testing efficacy of column fractions of C. velutina supernatants.

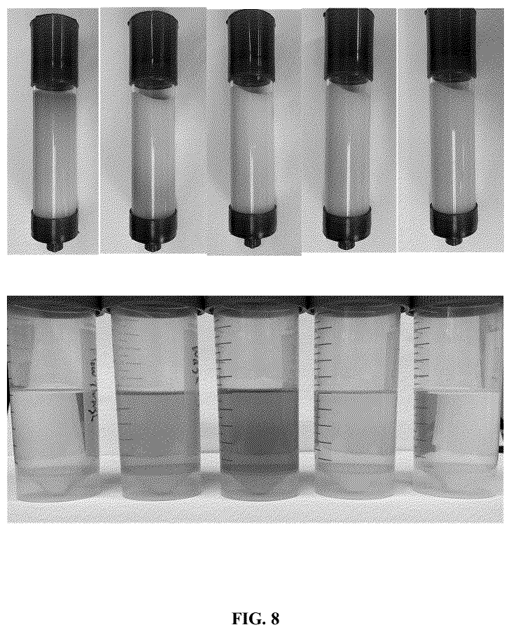

Three-week-old shake cultures (120 rpm, 20 C) of C. velutina (NRRL 68077) grown on potato dextrose broth were harvested. Filtration is required when using column separation due to the tiny, yeast-like spores that may interfere with column flow. Four flasks (100 ml liquid each) were filtered through Whatman #40 filter paper, followed by filtration through Nalgene 50 mm rapid flow filter units. Final filtrates were pooled, and an aliquot used to load a column (13×3 cm) packed with BioRad BioGel P-2 (8×3 cm), allowing for a 21 ml head volume to apply the filtrate and four subsequent water washes (FIG. 8).

Samples were syringe filter sterilized (0.45 micron) and added to Corning 24 count Cell Wells along at a 1:20 dilution (50 microliter sample, 0.9 ml potato dextrose broth and 50 microliters of an Alternaria solani spore suspension (approx. 1,000 spores final) or a 1:40 dilution (25 microliter sample, 0.925 ml potato dextrose broth and 50 microliter A. solani spore suspension). Fractionated samples were compared to crude culture filtrates for toxicity. The active fraction was primarily in water wash 1, which is the first elution after flow through of the original filtrate volume. This fraction was as active in suppressing Alternaria as equivalent volumes of the crude filtrate.

Samples were surveyed using 0.5 ml samples in a Jenway 7415 UV-Vis spectrophotometer in a UV range from 250 to 360 nanometers, where differences were observed between fractions. The active fraction eluting with the first water wash was differentiated by a peak at 310 nm, with absorbance values of 1.2, compared to absorbance of 0.25 for other fractions. The inactive fractions generally had sharp peaks between 250 and 300 nm and a broad curve from 300 to 360 nm, however overall absorbance was about 0.25 (data not shown). The high level of hydrophilicity has made characterization more challenging as organic solvents do not extract the activity.

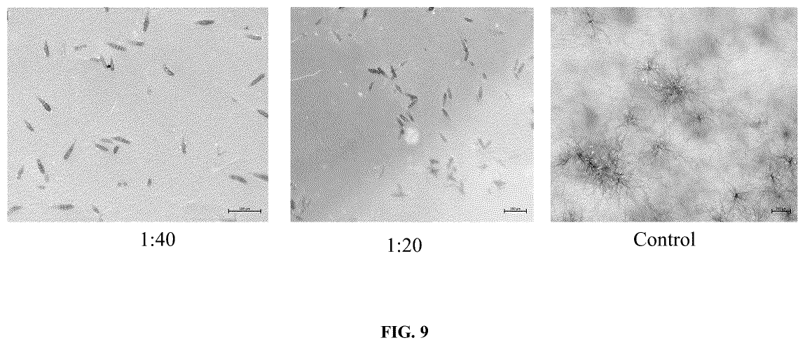

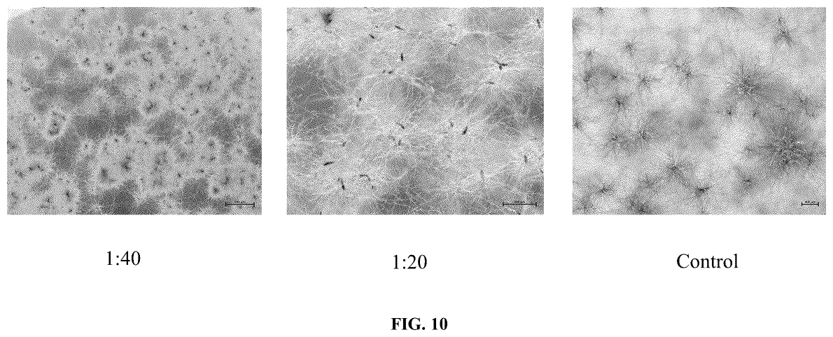

Samples of crude filtrate and water wash 1 both exhibit the same activity against spores of Alternaria solani. Immediate exposure followed by 48 hr incubation results in minimal levels of germination, and a lack of melanin production (FIG. 9). Exposure 24 hr after incubation, followed by an additional 24 hr incubation results in arrest of germ tube growth and a lack of melanization (FIG. 10).

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A composition comprising, a supernatant of an artificial liquid culture of Coniochaeta velutina strain NRRL 68077.

2. The composition of claim 1, wherein said supernatant is filtered.

3. The composition of claim 2, wherein the supernatant is substantially cell free.

4. The composition of claim 1, wherein the artificial liquid culture is potato dextrose broth.

5. The composition of claim 1, wherein the supernatant is not treated with an organic solvent.

6. The composition of claim 1, wherein the supernatant was collected from a culture at least 2-weeks old.

7. The composition of claim 1, wherein the supernatant was collected from a culture at least 3-weeks old.

8. A composition comprising, a water-soluble fraction of a supernatant of an artificial liquid culture of Coniochaeta velutina strain NRRL 68077.

9. A composition comprising, a water-soluble fraction of a supernatant of an artificial liquid culture of Coniochaeta velutina, wherein the water-soluble fraction comprises fungicidal activity against Alternaria solani, and wherein the non-water-soluble fraction does not comprise fungicidal activity against Alternaria solani.

10. The composition of claim 9, wherein the C. velutina is strain NRRL 68077.

11. The composition of claim 9, wherein the water-soluble fraction has not been contacted with methanol.

12. A composition comprising, a living plant and the composition of claim 1, claim 8 or claim 9.

13. A method of killing a fungal cell, comprising contacting an effective amount of the composition of claim 1, claim 8, or claim 9 to the fungal cell.

14. The method of claim 13, wherein the supernatant is diluted by at least 1:10.

15. The method of claim 13, wherein the fungal cell comprises the species Alternaria solani.
